(19) United States
(12) Patent Application Publication
(10) Pub. No.: US 2016/0168179 A1
(43) Pub. Date: Jun. 16, 2016

(54) CYCLOPOLYARYLENE METAL COMPLEX

(71) Applicant: National University Corporation Nagoya University, Nagoya-shi (JP)

(72) Inventors: Kenichiro Itami, Nagoya-shi (JP); Yasutomo Segawa, Nagoya-shi (JP); Natsumi Kubota, Nagoya-shi (JP)

(21) Appl. No.: 14/969,601

(22) Filed: Dec. 15, 2015

(30) Foreign Application Priority Data
Dec. 15, 2014 (JP) 2014-253322

(51) Int. Cl. C07F 11/00; C07F 5/04; C07C 67/39; C07F 7/08; C07C 69/78

(52) U.S. Cl. CPC C07F 11/00; C07F 7/0809; C07F 7/0827; C07C 69/78; C07C 67/39; C07F 5/04

(57) ABSTRACT

If a method for directly functionalizing cycloparaphenylene compounds is developed, such a method is expected to be applied to any cycloparaphenylene compound, thus theoretically enabling introduction of a functional group into all cycloparaphenylene compounds. Therefore, a primary object of the present invention is to provide a method for easily functionalizing cycloparaphenylene compounds directly. A cyclopolyarylene metal complex in which a metal tricarbonyl is coordinated to one benzene ring of a cyclopolyarylene compound is provided. The cyclopolyarylene metal complex is obtained by using a production method comprising the step of reacting a cyclopolyarylene compound with a metal compound represented by the following formula:

M(CO)₃Yₘ,

wherein M is a metal atom; Y is the same or different, and each represents a ligand; and m is an integer of 1 to 3.

### TECHNICAL FIELD

The present invention relates to a cyclopolyarylene metal complex and a method for producing the metal complex. The present invention also relates to a metal-substituted cyclopolyarylene compound and a functional-group-containing cyclopolyarylene compound, both obtained using the metal complex.

### BACKGROUND ART

Previously known nanostructures containing carbon atoms include carbon nanotubes made of a cylindrically rolled two-dimensional graphene sheet, cyclic carbon nanotubes containing such carbon nanotubes, and the like.

Carbon nanotubes have extremely high mechanical strength and high temperature resistance, and efficiently discharge electrons when voltage is applied. With these advantageous properties, carbon nanotubes are expected to be applied in various fields, including chemistry, electronics, and life sciences.

Known methods of producing carbon nanotubes include arc discharge, laser furnaces, chemical vapor deposition, and the like. However, these methods have a disadvantage in that they can only produce mixtures of carbon nanotubes with various diameters and lengths.

As a replacement for tubular nanostructures such as carbon nanotubes with a certain length derived from a continuous linkage of carbon atoms, recent studies have focused attention on cyclic nanostructures. For example, cycloparaphenylene (CPP) is a simple and beautiful molecule in which benzenes are linked at the para-positions to form a circle. Recent studies have revealed that cycloparaphenylene has a significantly distinctive structure and nature. In particular, since CPP has various diameters depending on the number of benzene rings it contains, and thus has various natures, if CPP is selectively produced, it has the potential to produce carbon nanotubes with various diameters. Therefore, the thoroughly selective production of CPP having different numbers of benzene rings has been desired. However, although a method for obtaining CPP as a mixture is known, the selective synthesis of CPP has been successful in only a few cases.

The present inventors succeeded in the synthesis of various cycloparaphenylene compounds through a method using a cyclic cycloparaphenylene precursor that contains a cyclohexane ring as a flexural portion (for example, Patent Literature 1 and 2, and Non-patent Literature 1).

However, although cycloparaphenylene compounds have a significantly distinctive structure and nature as described above, the introduction of a new function by adding a functional group to these compounds has not been developed. Since cycloparaphenylene compounds are highly symmetrical molecules and have many equivalent reaction sites (for example, [12]CPP, which has 12 benzene rings, has 48 equivalent reaction sites), it is difficult to introduce a desired number of functional groups at desired positions.

Nevertheless, synthesis of functionalized cycloparaphenylene compounds has the potential to lead to synthesis of various unique compounds, such as dimers of cycloparaphenylene compounds. Thus, there is a need for a method to introduce a desired number of functional groups into desired portions of a cycloparaphenylene compound.

In such a situation, only a method for newly synthesizing cyclic compounds using functional-group-containing monomers (bottom-up method) is known (for example, Patent Literature 3 and Non-patent Literature 2).

### CITATION LIST

### Patent Literature

PTL 1: WO2011/099588
PTL 2: WO2011/111719
PTL 3: WO2013/133386

### Non-Patent Literature

NPL 1: Takaba, H.; Omachi, H.; Yamamoto, Y.; Bouffard, J.; Itami, K. Angew. Chem. Int. Ed. 2009, 48, 6112
NPL 2: Ishii Y.; Matsuura S.; Segawa Y.; Itami K. Org. Lett. 2014, 16, 2174

### SUMMARY OF INVENTION

### Technical Problem

Since there are cycloparaphenylene compounds with various sizes, effective functionalization for each compound is required. The method of Patent Literature 3 and Non-patent Literature 2 (bottom-up method) is useful for obtaining such functionalized cycloparaphenylene compounds; however, it has been difficult to directly functionalize cycloparaphenylene compounds. If a method for directly functionalizing cycloparaphenylene compounds is developed, such a method is expected to be applied to any cycloparaphenylene compound, thus theoretically enabling introduction of a functional group into all cycloparaphenylene compounds. Therefore, a primary object of the present invention is to provide a method for easily functionalizing cycloparaphenylene compounds directly.

### Solution to Problem

The inventors of the present invention conducted extensive research to solve the above problems and found that use of a metal complex obtained by complexation of a benzene ring of a cycloparaphenylene compound with a predetermined metal makes it possible to easily functionalize a cycloparaphenylene compound directly. In addition, since only the moiety coordinated to the metal is highly reactive in the metal complex, it is also possible to easily introduce a desired number of functional groups into a desired portion of a cycloparaphenylene compound through a deprotonation reaction, a reaction with an electrophile, or the like. The inventors conducted further research based on this finding and have accomplished the present invention. Specifically, the present invention encompasses the following features.

Item 1. A cyclopolyarylene metal complex in which a metal tricarbonyl is coordinated to one benzene ring of a cyclopolyarylene compound.

Item 2. The cyclopolyarylene metal complex according to Item 1, wherein the cyclopolyarylene compound is a cyclic compound in which at least one member selected from the group consisting of bivalent aromatic hydrocarbon groups and derivative groups thereof are continuously bonded.

Item 3. The cyclopolyarylene metal complex according to Item 1 or 2, wherein the metal constituting the metal tricarbonyl is chromium, molybdenum, tungsten, iron, ruthenium, osmium, manganese, or rhenium.

Item 4. A method for producing the cyclopolyarylene metal complex according to any one of Items 1 to 3, the method comprising the step of (I) reacting a cyclopolyarylene compound with a metal compound represented by Formula (2):

M(CO)₃Yₘ,

wherein M is a metal atom; Y is the same or different, and each represents a ligand; and m is an integer of 1 to 3.

Item 5. The method according to Item 4, wherein the step (I) is performed in the presence of an ether solvent or a hydrocarbon solvent.

Item 6. A metal-substituted cyclopolyarylene compound in which a metal atom is bonded to one carbon atom of one benzene ring of a cyclopolyarylene compound.

Item 7. The metal-substituted cyclopolyarylene compound according to Item 6, wherein the metal atom is an alkali metal atom.

Item 8. A method for producing a metal-substituted cyclopolyarylene compound, the method comprising the step of (II) reacting the cyclopolyarylene metal complex according to any one of Items 1 to 3 or a cyclopolyarylene metal complex obtained by the method according to Item 4 or 5 with a metal compound.

Item 9. The method according to Item 8, wherein the metal compound is an alkali metal compound.

Item 10. The method according to Item 8 or 9, wherein the metal compound is an alkyllithium.

Item 11. A functional-group-containing cyclopolyarylene compound in which a boronic acid group or an ester thereof, a silyl group, a carboxy group or an ester thereof, or a formyl group is bonded to one carbon atom of one benzene ring of a cyclopolyarylene compound.

Item 12. A method for producing a functional-group-containing cyclopolyarylene compound, the method comprising the step of (III) reacting the metal-substituted cyclopolyarylene compound according to Item 6 or 7 or a metal-substituted cyclopolyarylene compound obtained by the method according to any one of Items 8 to 10 with an electrophile.

### Advantageous Effects of Invention

The metal complex of the present invention shows that various cycloparaphenylene compounds can be complexed by reacting them with a specific metal compound. In this complexation, only one benzene ring of the cycloparaphenylene compound can be complexed.

Use of the metal complex of the present invention makes it possible to easily perform, for example, direct metalation of cycloparaphenylene compounds (synthesis of metal-substituted cyclopolyarylene compounds) and direct functionalization of cycloparaphenylene compounds (synthesis of functional-group-containing cyclopolyarylene compounds), both of which were previously difficult.

In the metal complex of the present invention, only the moiety coordinated to the metal is highly reactive; therefore, into a desired portion of a cycloparaphenylene compound can be introduced a desired number of metals (synthesis of metal-substituted cyclopolyarylene compounds), functional groups (synthesis of functional-group-containing cyclopolyarylene compounds), or the like through a deprotonation reaction, a reaction with an electrophile, or the like. In other words, it is possible to achieve complexation with various metals, metalation, functionalization, or the like for various cycloparaphenylene compounds. Thus, the present invention is useful because it is highly versatile.

In particular, the present invention enables complexation, metalation, functionalization, and the like of cycloparaphenylene compounds and like cyclic compounds that are highly symmetrical and have many equivalent reaction sites (preferably introduction of a desired number of complexes, metals, functional groups, or the like into a desired portion).

It is expected that use of the metal complex of the present invention, the metal-substituted cyclopolyarylene compound of the present invention, or the functional-group-containing cyclopolyarylene compound of the present invention enables synthesis of cycloparaphenylene dimmers. Also expected is synthesis of carbon nanobelts in which all corresponding carbon atoms of two molecules of a cycloparaphenylene compound are bonded together. Unlike previously known methods, the method of the present invention allows complexation with various metals, metalation, functionalization, and the like for various cycloparaphenylene compounds; therefore, synthesis of various cycloparaphenylene dimmers, carbon nanobelts, carbon nanotubes, cyclic carbon nanotubes, and the like are also anticipated. The cyclopolyarylene compound of the present invention is thus expected to be applied in various fields, including chemistry, electronics, and life sciences.

### DESCRIPTION OF EMBODIMENTS

### [1] Cyclopolyarylene Metal Complex

The cyclopolyarylene metal complex of the present invention is a cyclopolyarylene metal complex in which a metal tricarbonyl is coordinated to one benzene ring of a cyclopolyarylene compound.

### 1-1. Cyclopolyarylene Compound

In the present invention, the cyclopolyarylene compound is a cyclic compound in which multiple arylene groups form a cyclic structure via single bonds and in which no complexes, metals, or substituents such as functional groups are introduced. More specifically, such a compound is a cyclic compound represented by Formula (A):

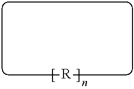

wherein R is the same or different, and each represents an arylene group; and n is an integer of 5 to 30.

In Formula (A), R is an arylene group. Specifically, R is a bivalent group containing an aromatic ring, which is obtained by eliminating a hydrogen atom from each of two carbon atoms of the aromatic ring. Each R may be the same or different.

In addition to benzene rings, examples of aromatic rings include rings resulting from the condensation of multiple benzene rings (benzene-condensed rings), rings resulting from the condensation of benzene and other rings, and the like (hereafter, these rings resulting from the condensation of multiple benzene rings and rings resulting from the condensation of benzene and other rings may be collectively referred to as “condensed rings”). Examples of condensed rings include a pentalene ring, indene ring, naphthalene ring, anthracene ring, tetracene ring, pentacene ring, pyrene ring, perylene ring, triphenylene ring, azulene ring, heptalene ring, biphenylene ring, indacene ring, acenaphthylene ring, fluorene ring, phenalene ring, phenanthrene ring, and the like.

R is preferably a bivalent group that contains a 6-membered aromatic ring or a 6-membered heterocyclic aromatic ring among the above rings, and that has binding sites at the para-positions.

Further, the aromatic ring of R is preferably a monocyclic or condensed ring. A monocyclic ring is more preferable.

Among these, R is preferably a phenylene group (in particular, 1,4-phenylene group), a naphthylene group (in particular, 1,5-naphthylene group or 2,6-naphthylene group), or the like. A phenylene group (in particular, 1,4-phenylene group) is more preferable.

In the cyclic compound of the present invention, n, i.e., the number of arylene groups is an integer of 5 to 30, preferably an integer of 5 to 20, more preferably an integer of 5 to 18, even more preferably an integer of 5 to 16 or 18, and particularly preferably an integer of 6 to 15.

The cyclopolyarylene compound used in the present invention is preferably a cycloparaphenylene compound in which all of the organic ring groups are phenylene groups (in particular, 1,4-phenylene groups).

Among cyclopolyarylene compounds used in the present invention, a cycloparaphenylene compound consisting of 1,4-phenylene groups is, for example, a compound represented by Formula (A1):

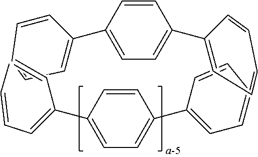

wherein a is an integer of 6 or more.

### 1-2. Method for Producing Cyclopolyarylene Compound

The cyclopolyarylene compound used in the present invention can be synthesized by using a known method or can be a commercially available product.

For example, the cyclopolyarylene compound used in the present invention can be produced according to the method described in Patent Literature 1, 2, or 3; Jasti, R. et al., J. Am. Chem. Soc., 2008, 130(52), 17646; Itami, K. et al., Angew. Chem. Int. Ed., 2009, 48, 6112 (Non-patent Literature 1); Itami, K. et al., Angew. Chem. Int. Ed., 2010, 49, 10202; Yamago, S. et al., Angew. Chem. Int. Ed., 2009, 49, 75; Jasti, R. et al., Nature Chemistry, 2014, 6, 404; Jasti, R. et al., J. Org. Chem., 2012, 77, 10473; Itami, K. et al., Chem. Sci. 2012, 3, 2340; or the like, or a method analogous to this method. If necessary, cyclopolyarylene compounds having various numbers of rings can be obtained by using various methods.

### 1-3. Metal Tricarbonyl

In the cyclopolyarylene metal complex of the present invention, there are no particular limitations on the metal constituting a metal tricarbonyl coordinated to the cyclopolyarylene compound described above. Examples of metals include chromium, molybdenum, tungsten, iron, ruthenium, osmium, manganese, rhenium, and the like. Among these, chromium, molybdenum, tungsten, and the like are preferable in terms of reactivity. The metal may be appropriately selected according to physical properties required for the cyclopolyarylene metal complex.

In the cyclopolyarylene metal complex of the present invention, only one metal tricarbonyl is coordinated to one benzene ring of the cyclopolyarylene compound. More specifically, the cyclopolyarylene metal complex of the present invention has a bivalent group represented by Formula (1):

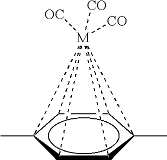

wherein M is a metal atom; and six dotted lines connecting M and the six carbon atoms of a benzene ring, and three dotted lines connecting M and three CO each represent a coordinate bond.

Examples of the metal atom represented by M in Formula (1) include chromium, molybdenum, tungsten, iron, ruthenium, osmium, manganese, rhenium, and the like. Among these, chromium, molybdenum, tungsten, and the like are preferable in terms of reactivity.

In the cyclopolyarylene metal complex of the present invention, groups other than the above bivalent group are preferably all 1,4-phenylene groups.

Specifically, the cyclopolyarylene metal complex of the present invention is preferably a compound represented by Formula (6):

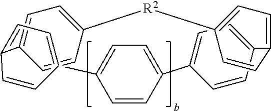

wherein R² is a bivalent group represented by Formula (1); and b is an integer of 0 to 25.

In Formula (6), b may be appropriately set according to required properties, and is preferably an integer of 0 to 25, more preferably an integer of 0 to 15, even more preferably an integer of 0 to 13, particularly preferably an integer of 0 to 11 or 13, and most preferably an integer of 1 to 10.

As stated above, the present invention makes it possible to coordinate a metal tricarbonyl to only one benzene ring; therefore, only one portion of the cyclopolyarylene compound can be functionalized.

### [2] Method for Producing Cyclopolyarylene Metal Complex

Although there are no particular limitations, the cyclopolyarylene metal complex of the present invention can be obtained by using a production method comprising the step of (I) reacting a cyclopolyarylene compound with a metal compound represented by Formula (2):

M(CO)₃Yₘ,

wherein M is a metal atom; Y is the same or different, and each represents a ligand; and m is an integer of 1 to 3.

As the cyclopolyarylene compound, the cyclopolyarylene compound described above can be used.

Examples of the metal atom represented by M in Formula (2) include chromium, molybdenum, tungsten, iron, ruthenium, osmium, manganese, rhenium, and the like. Among these, chromium, molybdenum, tungsten, and the like are preferable in terms of reactivity.

In Formula (2), the ligand represented by Y is not particularly limited as long as it can be coordinated to the metal atom represented by M (such as chromium, molybdenum, tungsten, iron, ruthenium, osmium, manganese, or rhenium).

Examples of ligands include carbonyl (CO), isocyanide, arenes, olefins, pyridines, amines, phosphines, carbenes, nitriles, hydrogen (hydride; H⁻), halogen, lower alkoxy, boron-containing ligands, phosphorus-containing ligands, antimony-containing ligands, arsenic-containing ligands, sulfonic-acid-based ligands, sulfate, perchlorate, nitrate, bis(triflyl)imide, tris(triflyl)methane, bis(triflyl)methane, carboxylates, and the like. The ligands are preferably all carbonyl groups.

Examples of nitriles as the ligand represented by Y in Formula (2) include benzonitrile, acetonitrile, propionitrile, and the like.

Examples of halogen atoms as the ligand represented by Y in Formula (2) include fluorine, chlorine, bromine, and iodine.

In Formula (2), m is an integer of 1 to 3, and preferably 3.

The metal compound represented by Formula (2) may be a known or commercially available metal compound. The ligands, i.e., carbon monoxide (CO) and Y, may be coordinated in advance or may be coordinated in the system. Specifically, in the coupling reaction of the present invention, a metal compound in which carbon monoxide (CO) and Y are coordinated may be used, or one or more predetermined ligand compounds and a predetermined metal compound may be used.

Such metal compounds may be used singly or in a combination of two or more. The metal compound is preferably selected according to physical properties required for the cyclopolyarylene metal complex of the present invention.

The amount of the metal compound varies depending on the type of metal it contains and, for example, is generally preferably about 0.5 to about 10 mol, and more preferably about 1 to about 3 mol, per mol of the cyclopolyarylene compound that is a substrate. When the metal compound is synthesized in the system, it is preferable that the amount of the metal compound in the system be adjusted within the above range.

It is preferable that step (I) be generally performed in the presence of a reaction solvent. Examples of reaction solvents include chain ethers such as dimethoxyethane, diisopropyl ether, di-n-butyl ether, and tert-butyl methyl ether; cyclic ethers such as dioxane and tetrahydrofuran; aliphatic hydrocarbons such as hexane, cyclohexane, and heptane; aromatic hydrocarbons such as benzene, toluene, xylene, and chlorobenzene; and the like. These may be used singly or in a combination of two or more. Among these, in the present invention, ether solvents (such as chain ethers and cyclic ethers) or hydrocarbon solvents (aliphatic hydrocarbons and aromatic hydrocarbons) are preferable. Ether solvents (such as chain ethers and cyclic ethers) are more preferable, and di-n-butyl ether, tetrahydrofuran, and the like are more preferable.

When the reaction solvent is used, the concentration of the cyclopolyarylene compound as a substrate in the reaction solvent is not particularly limited, and is preferably 1 to 10 mM.

The reaction temperature in the above reaction is generally selected from a temperature range of not less than 0° C. and not more than the boiling point of the reaction solvent. The reaction time may be a period of time sufficient for the reaction to proceed.

The reaction atmosphere is not particularly limited; an inert gas atmosphere, such as an argon gas atmosphere or a nitrogen gas atmosphere, is preferable. It is also possible to use air atmosphere.

After the reaction, a purification step may be performed as necessary. In the purification step, general post-treatment steps, such as solvent removal, washing, and chromatography separation, may be performed.

### [3] Metal-Substituted Cyclopolyarylene Compound

In the metal-substituted cyclopolyarylene compound of the present invention, a metal atom is bonded to one carbon atom of one benzene ring of a cyclopolyarylene compound.

The metal atom bonded to one carbon atom of one benzene ring of a cyclopolyarylene compound is not particularly limited. Examples include alkali metal atoms, alkaline earth metal atoms, and the like. Alkali metals are preferable. Lithium atom, sodium atom, and the like are more preferable, and lithium atom is even more preferable.

Specifically, the metal-substituted cyclopolyarylene compound of the present invention is preferably, for example, a compound represented by Formula (7):

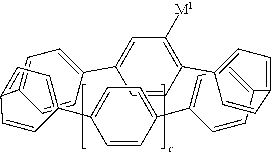

wherein M¹ is a metal atom; and c is an integer of 0 or more.

The metal atom represented by M¹ in Formula (7) is not particularly limited. Examples include alkali metal atoms, alkaline earth metal atoms, and the like. Alkali metals are preferable. Lithium atom, sodium atom, and the like are more preferable, and lithium atom is even more preferable.

In Formula (7), c may be appropriately set according to required properties; c is preferably an integer of 0 to 25, more preferably an integer of 0 to 15, even more preferably an integer of 0 to 13, particularly preferably an integer of 0 to 11 or 13, and most preferably an integer of 1 to 10.

The metal-substituted cyclopolyarylene compound can also be obtained as a synthetic intermediate when a functional-group-containing cyclopolyarylene compound is obtained from the cyclopolyarylene metal complex described above.

### [4] Method for Producing Metal-Substituted Cyclopolyarylene Compound

The metal-substituted cyclopolyarylene compound of the present invention can be produced, for example, by using a method comprising the step of (II) reacting the cyclopolyarylene metal complex of the present invention with a metal compound.

The metal compound is not particularly limited and is preferably an organic alkali metal compound. Examples include organic lithium compounds, organic sodium compounds, and the like. Organic lithium compounds are particularly preferable. Examples of organic lithium compounds include organic monolithium compounds, organic dilithium compounds, organic polylithium compounds, and the like.

Specific examples of organic lithium compounds include alkyllithiums, such as ethyllithium, n-propyllithium, isopropyllithium, n-butyllithium, sec-butyllithium, tert-butyllithium, pentyllithium, and hexyllithium; cycloalkyllithiums, such as cyclohexyllithium; aryllithiums, such as phenyllithium; hexamethylene dilithium, cyclopentadienyl lithium, indenyl lithium, 1,1-diphenyl-n-hexyllithium, 1,1-diphenyl-3-methylpentyllithium, lithium naphthalene, butadienyl dilithium, isopropenyl dilithium, m-diisoprenyl dilithium, 1,3-phenylene-bis-(3-methyl-1-phenylpentylidene)bislithium, 1,3-phenylene-bis-(3-methyl-1,[4-methylphenyl]pentylidene)bislithium, 1,3-phenylene-bis-(3-methyl-1,[4-dodecylphenyl]pentylidene)bislithium, 1,1,4,4-tetraphenyl-1,4-dilithio butane, polybutadienyl lithium, polyisoprenyl lithium, polystyrene-butadienyl lithium, polystyrenyl lithium, polyethylenyl lithium, poly-1,3-cyclohexa dienyl lithium, polystyrene-1,3-cyclohexadienyl lithium, polybutadiene-1,3-cyclohexadienyl lithium, and the like. These may be used singly or in a combination of two or more. Among these, in terms of the yield, organic monolithium compounds are preferable, alkyllithiums, cycloalkyllithiums, aryllithiums, and the like are more preferable, and ethyllithium, n-propyllithium, isopropyllithium, n-butyllithium, sec-butyllithium, tert-butyllithium, pentyllithium, hexyllithium, cyclohexyllithium, phenyllithium, and the like are even more preferable.

The amount of the metal compound is not particularly limited. In terms of the yield, the amount of the metal compound is generally preferably 2 to 50 mol, more preferably 3 to 30 mol, and even more preferably 5 to 20 mol, per mol of the cyclopolyarylene metal complex of the present invention.

The above reaction is generally performed in the presence of a reaction solvent. Examples of reaction solvents include ethers, such as diethyl ether, tetrahydrofuran, dioxane, dimethoxyethane, and diisopropyl ether; hydrocarbon solvents, such as hexane and pentane; and the like. These may be used singly or in a combination of two or more. Among these, ethers (such as tetrahydrofuran and diethyl ether) are preferable in the present invention.

When the reaction solvent is used, the concentration of the cyclopolyarylene metal complex of the present invention in the reaction solvent is not particularly limited and is preferably 1 to 15 mM.

The reaction temperature is generally selected from a temperature range of not less than −100° C. and not more than the boiling point of the reaction solvent. The reaction time may be a period of time sufficient for the reaction to proceed.

The reaction atmosphere is not particularly limited; an inert gas atmosphere, such as an argon gas atmosphere or a nitrogen gas atmosphere, is preferable. It is also possible to use air atmosphere.

After the reaction step, a purification step may be performed as necessary. In the purification step, general post-treatment steps, such as solvent removal, washing, and chromatography separation, may be performed.

### [5] Functional-Group-Containing Cyclopolyarylene Compound

In the functional-group-containing cyclopolyarylene compound of the present invention, a boronic acid group or an ester thereof, a silyl group, a carboxy group or an ester thereof, or a formyl group is bonded to one carbon atom of one benzene ring of a cyclopolyarylene compound.

The functional group bonded to one carbon atom of one benzene ring of a cyclopolyarylene compound is, for example, a boronic acid group or an ester thereof, a silyl group, a carboxy group or an ester thereof, a formyl group, or the like. The functional group is preferably a carboxy group or an ester thereof, or a formyl group.

The boronic acid group or an ester thereof is, for example, preferably a group represented by the formula below:

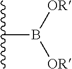

wherein R′ is the same or different, and each represent a hydrogen atom or a lower alkyl group (in particular, C₁₋₁₀ alkyl group), and R′ may be bonded to each other to form a ring with the adjacent —O—B—O—.

R′ in the boronic acid group or an ester thereof is a hydrogen atom or an alkyl group. The alkyl group preferably has 1 to 10, more preferably 1 to 8, even more preferably 1 to 5 carbon atoms. Further, in the above formula, the two R′ may be the same or different. When R′ represents an alkyl group, the carbon atoms of the alkyl groups may be bonded to form a ring with the boron atom and the oxygen atoms.

Examples of such a boronic acid group or an ester thereof include groups represented by the formulae below:

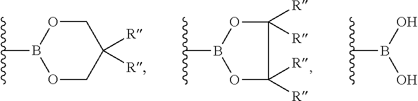

wherein R″ is the same or different, and each represents a hydrogen atom or a lower alkyl group (in particular, C₁₋₁₀ alkyl group). The boronic acid group or an ester thereof is particularly preferably a group represented by the formula below:

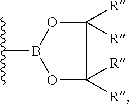

Examples of the silyl group include a trimethylsilyl group, a triethylsilyl group, a t-butyldimethylsilyl group, and the like.

Examples of the carboxy group or an ester thereof include, in addition to a carboxy group, esters of a carboxy group, such as a carboxymethyl group and a carboxyethyl group.

The functional group may be appropriately selected according to required properties.

Specifically, the functional-group-containing cyclopolyarylene compound of the present invention is, for example, preferably a compound represented by Formula (8):

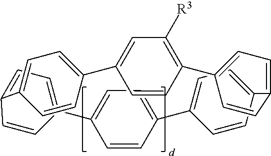

wherein R³ is a functional group (such as a boronic acid group or an ester thereof, a silyl group, a carboxy group or an ester thereof, or a formyl group); and d is an integer of 1 or more.

In Formula (8), d may be appropriately set according to required properties; d is preferably an integer of 1 or more, more preferably an integer of 1 to 50, even more preferably an integer of 2 to 30, and particularly preferably an integer of 3 to 20.

### [6] Method for Producing Functional-Group-Containing Cyclopolyarylene Compound

The functional-group-containing cyclopolyarylene compound of the present invention can be produced, for example, by using a method comprising the step of (III) reacting the metal-substituted cyclopolyarylene compound of the present invention with an electrophile.

After the cyclopolyarylene metal complex of the present invention is reacted with a metal compound according to step (II) described above, an electrophile may be added as is.

The electrophile is not particularly limited. Examples of electrophiles include esterifying agents, borylating agents, substituted silylating agents, acylating or formylating agents, and the like.

Examples of esterifying agents include methyl iodoformate, ethyl iodoformate, methyl iodoacetate, ethyl iodoacetate, methyl bromoformate, ethyl bromoformate, methyl bromoacetate, ethyl bromoacetate, methyl chloroformate, ethyl chloroformate, methyl chloroacetate, ethyl chloroacetate, and the like. Among these, methyl chloroformate and the like are preferable.

Examples of borylating agents include methoxyboronic acid, ethoxyboronic acid, methoxyboronic acid pinacol ester, ethoxyboronic acid pinacol ester, and the like. Among these, methoxyboronic acid pinacol ester and the like are preferable.

Examples of substituted silylating agents include substituted silyl iodides, such as iodotrimethylsilane, iodotriethylsilane, iodotributylsilane, iodotricyclohexylsilane, and iodotriphenylsilane; substituted silyl bromides, such as bromotrimethylsilane, bromotriethylsilane, bromotributylsilane, bromotricyclohexylsilane, and bromotriphenylsilane; substituted silyl chlorides, such as chlorotrimethylsilane, chlorotriethylsilane, chlorotributylsilane, chlorotricyclohexylsilane, and chlorotriphenylsilane; substituted silyl mesylates, such as mesylate trimethylsilane, mesylate triethylsilane, mesylate tributylsilane, mesylate tricyclohexylsilane, and mesylate triphenylsilane; substituted silyl tosylates, such as tosylate trimethylsilane, tosylate triethylsilane, tosylate tributylsilane, tosylate tricyclohexylsilane, and tosylate triphenylsilane; substituted silyl triflates, such as triflate trimethylsilane, triflate triethylsilane, triflate tributylsilane, triflate tricyclohexylsilane, and triflate triphenylsilane; and the like. Among these, substituted silyl chlorides are preferable. Chlorotrimethylsilane and the like are more preferable.

The acylating or formylating agents may have a linear, branched, or cyclic structure, and may have one or more substituent. The acylating or formylating agents generally have about 1 to about 20 carbon atoms. Specific examples of acylating or formylating agents include N,N-dimethylformamide, N,N-diethylformamide, and the like. Among these, N,N-dimethylformamide and the like are preferable.

These may be used singly or in a combination of two or more.

The amount of the electrophile is not particularly limited. In terms of the yield, the amount of the electrophile is generally preferably 1 to 500 mol, more preferably 1 to 300 mol, and even more preferably 1 to 200 mol, per mol of the metal-substituted cyclopolyarylene compound of the present invention.

The reaction described above is generally performed in the presence of a reaction solvent. Examples of reaction solvents include ethers such as diethyl ether, tetrahydrofuran, dioxane, dimethoxyethane, and diisopropyl ether; hydrocarbon solvents, such as hexane and pentane; and the like. These may be used singly or in a combination of two or more. Among these, ethers (such as tetrahydrofuran and diethyl ether) are preferable in the present invention. When the reaction is performed continuously after the above-described step (II), the same solvent can be used. However, the reaction intermediate between the starting materials and the functional-group-containing cyclopolyarylene compound may have low solubility in the solvent used. In this case, another solvent may be added in advance or during the reaction.

When the reaction solvent is used, the concentration of the metal-substituted cyclopolyarylene compound of the present invention in the reaction solvent is not particularly limited and may be similar to the concentration of the cyclopolyarylene metal complex in the reaction solvent in step (II).

The reaction temperature is generally selected from a temperature range of not less than −100° C. and not more than the boiling point of the reaction solvent. The reaction time may be a period of time sufficient for the reaction to proceed.

The reaction atmosphere is not particularly limited; an inert gas atmosphere, such as an argon gas atmosphere or a nitrogen gas atmosphere, is preferable. It is also possible to use air atmosphere.

After the reaction step, a purification step may be performed as necessary. In the purification step, general post-treatment steps, such as solvent removal, washing, and chromatography separation, may be performed.

After the functional-group-containing cyclopolyarylene compound of the present invention is produced as described above, the functional group can be replaced by another functional group by a known method.

### EXAMPLES

The present invention is described in detail below with reference to Examples, but is not limited to these.

Unless otherwise noted, all materials, including dry solvent were obtained from commercial suppliers and used without further purification. [9]CPP was synthesized according to an already published document (WO2011/111719). However, tetrahydrofuran (THF) and dibutyl ether were purified by passing through a solvent purification system (glass contour). All the reactions were performed using reagent-grade solvents under air. Ni(cod)₂ was synthesized according to an already published document.

Thin-layer chromatography (TLC) was performed using E. Merck silica gel 60 F254 precoated plates (0.25 mm). The chromatogram was analyzed with a UV lamp (254 nm and 365 nm). Flash column chromatography was performed using E. Merck silica gel 60 (230-400 mesh). Preparative thin-layer chromatography (PTLC) was performed using Wako-gel® B5-F silica coated plates (0.75 mm). High-resolution mass spectra (HRMS) were performed with a Thermo Fisher Scientific Exactive. Nuclear magnetic resonance (NMR) spectra were recorded with a JEOL JNM-ECA-600 (¹H 600 MHz, ¹³C 150 MHz) spectrometer. Chemical shifts for ¹H NMR are expressed in parts per million (ppm) relative to CHCl₃ (δ7.26 ppm), CHDCl₂ (δ5.32 ppm), DMSO-d₅ (δ2.50 ppm), or THF-d₇ (δ1.72 ppm). Chemical shifts for ¹³C NMR are expressed in parts per million (ppm) relative to CDCl₃ (δ77.0 ppm), CD₂Cl₂ (δ53.8 ppm), DMSO-d₆ (δ39.5 ppm), or THF-d₈ (δ7.2 ppm). Data are reported in the following order: chemical shift, multiplicity (s=singlet, d=doublet, dd=doublet of doublets, t=triplet, m=multiplet), coupling constant (Hz), and integration.

### Example 1

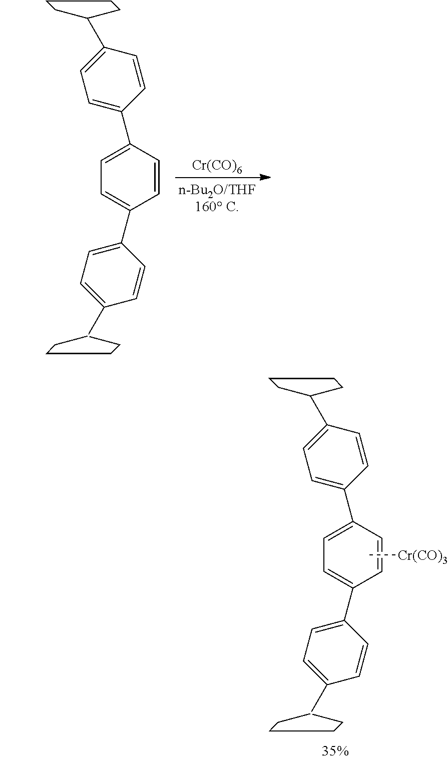

A magnetic stirring bar was placed in a J. Young® Schlenk flask, and [9]CPP (5.0 mg, 7.30 μmol), Cr(CO)₆ (1.7 mg, 7.52 μmol), dibutyl ether (0.9 mL), and THF (0.1 mL) were added to the flask. The reaction mixture was stirred at 160° C. for 10 hours in the dark and concentrated under reduced pressure. The crude product was purified by silica gel column chromatography (hexane/CHCl₃). As a result, the desired chromium-[9]CPP was obtained as an orange solid (2.1 mg, 35%).

1H NMR (600 MHz, CDCl₃) δ 5.46 (s, 4H), 7.40 (d, J=9.0, 4H), 7.56-7.52 (m, 28H). HRMS (ESI) m/z calcd for C₅₇H₃₆O₃CrCl [M.Cl]⁻: 855.1754. found 855.1782.

### Example 2

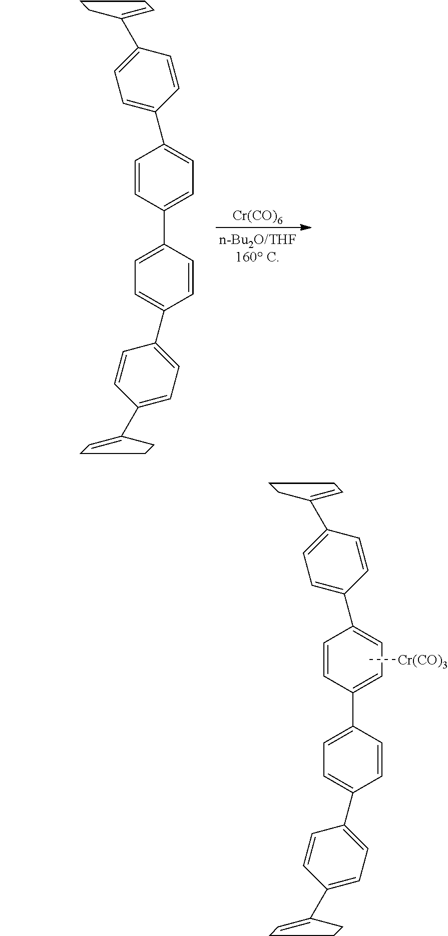

A magnetic stirring bar was placed in a J. Young® Schlenk flask, and [12]CPP (10.0 mg, 11.0 μmol), Cr(CO) (2.5 mg, 11.0 μmol), dibutyl ether (6.8 mL), and THF (0.8 mL) were added to the flask. The reaction mixture was stirred at 160° C. for 2 hours in the dark and concentrated under reduced pressure to give a crude product.

¹H NMR (600 MHz, CDCl₃) δ 5.61 (s, 4H). HRMS (ESI) m/z calcd for C₇₅H₄₉O₃Cr [MH]⁺: 1049.3081. found 1049.3106.

### Example 3

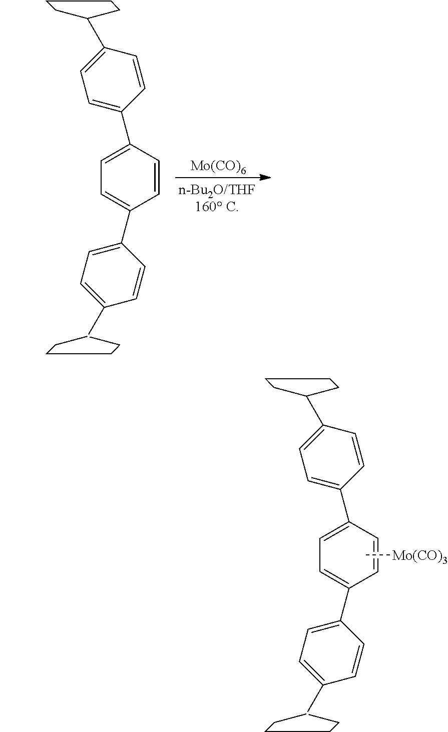

A magnetic stirring bar was placed in a J. Young® Schlenk flask, and [9]CPP (5.0 mg, 7.30 μmol), Mo(CO)₆ (30.0 mg, 114 μmol), dibutyl ether (1.8 mL), and THF (0.2 mL) were added to the flask. The reaction mixture was stirred at 160° C. for 1 hour in the dark and concentrated under reduced pressure to give a crude product.

¹H NMR (600 MHz, CDCl₃) δ 5.72 (s, 4H), HRMS (ESI) m/z calcd for C₅₇H₃₆O₃Mo [M.]⁺: 866.1728. found 866.1748.

### Example 4

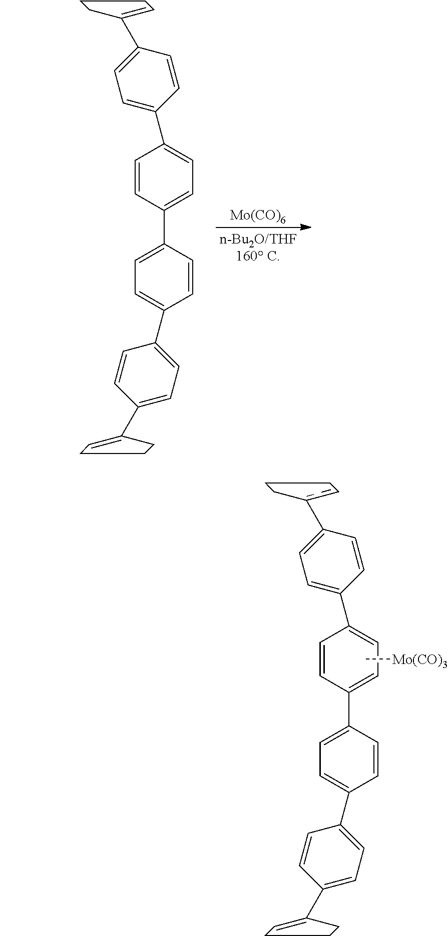

A magnetic stirring bar was placed in a J. Young® Schlenk flask, and [12]CPP (2.5 mg, 2.74 μmol), Mo(CO)₆ (30.0 mg, 114 μmol), dibutyl ether (0.9 mL), and THF (0.1 mL) were added to the flask. The reaction mixture was stirred at 160° C. for 1 hour in the dark and concentrated under reduced pressure to give a crude product.

¹H NMR (600 MHz, CDCl₃) δ 5.86 (s, 4H). HRMS (ESI) m/z calcd for C₇₅H₄₈O₃Mo [M.]⁺: 1094.2652. found 1094.2661.

### Example 5

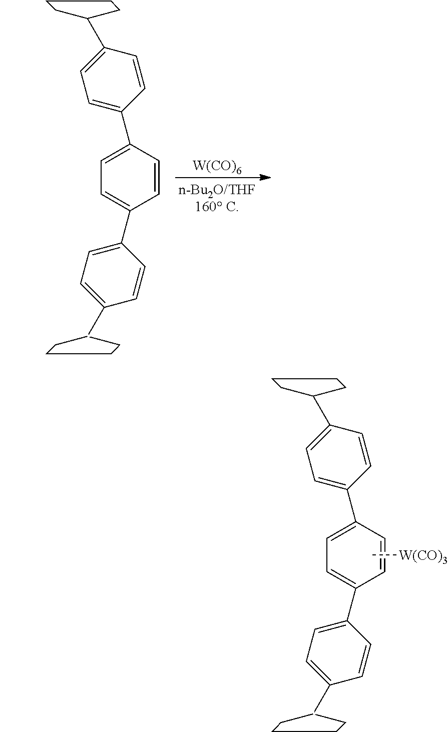

A magnetic stirring bar was placed in a J. Young® Schlenk flask, and [9]CPP (5.0 mg, 7.30 mol), W(CO)₆ (4.0 mg, 11.4 μmol), dibutyl ether (1.8 mL), and THF (0.2 mL) were added to the flask. The reaction mixture was stirred at 160° C. for 1 hour in the dark and concentrated under reduced pressure to give a crude product.

¹H NMR (600 MHz, CDCl₃) δ 5.56 (s, 4H). HRMS (ESI) m/z calcd for C₅₇H₃₆O₃W [M.]⁺: 952.2178. found 952.2155.

### Example 6

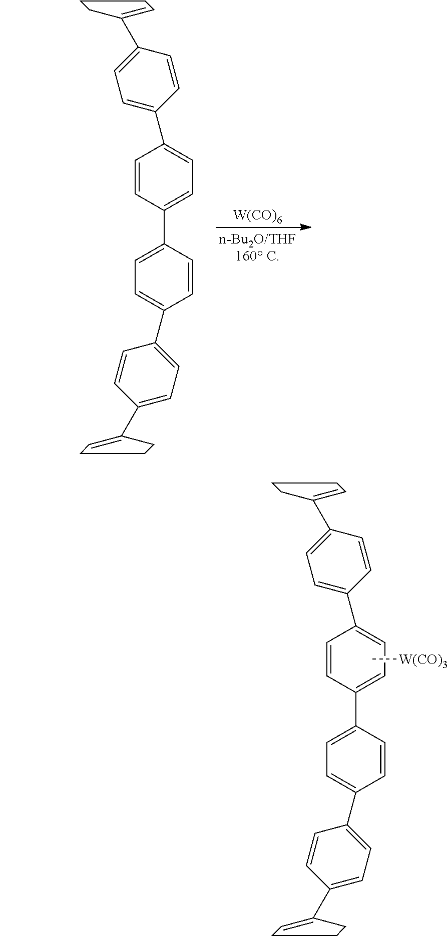

A magnetic stirring bar was placed in a J. Young® Schlenk flask, and [12]CPP (2.5 mg, 2.74 μmol), W(CO)₆ (70 mg, 199 μmol), dibutyl ether (2.7 mL), and THF (0.3 mL) were added to the flask. The reaction mixture was stirred at 160° C. for 1 hour in the dark and concentrated under reduced pressure to give a crude dark and concentrated under reduced pressure to give a crude product.

¹H NMR (600 MHz, CDCl₃) δ 5.69 (s, 4H). HRMS (ESI) m/z calcd for C₇₅H₄₉O₃W [MH]⁺: 1181.3199. found 1181.3179.

### Example 7

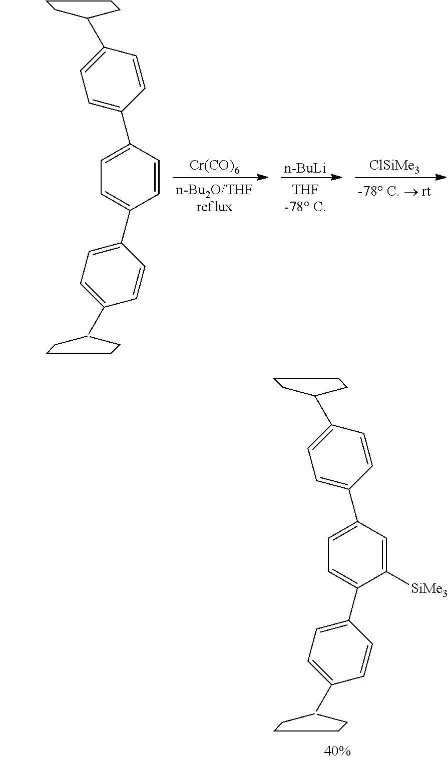

A magnetic stirring bar was placed in a J. Young® Schlenk flask, and [9]CPP (20.0 mg, 29.2 μmol), Cr(CO)₆ (7.2 mg, 31.7 μmol), dibutyl ether (9.0 mL), and THF (1.0 mL) were added to the flask. The reaction mixture was stirred at 160° C. for 1 hour in the dark and concentrated under reduced pressure. After the obtained product was dissolved in THF (5.0 mL), a hexane solution of 0.4 M n-butyllithium (150 μL, 60 μmol) was slowly added at −78° C. The reaction mixture was stirred for 30 minutes in the dark (at this point, lithiated [9]CPP was obtained). Thereafter, chlorotrimethylsilane (100 μL, 780 μmol) was added to the reaction mixture, and the resulting reaction mixture was warmed to room temperature and stirred for 1 hour in the dark. The reaction mixture was quenched with water and exposed to air and room light to perform decomplexation for 24 hours. The obtained reaction mixture was concentrated under reduced pressure, and the crude product was purified by silica gel column chromatography (hexane/CHCl₃). As a result, the desired trimethylsilyl-[9]CPP was obtained as a yellow solid (8.8 mg, 40%), and the starting material [9]CPP was recovered (9.1 mg, 46%).

¹H NMR (600 MHz, CDCl₃) δ 0.36 (s, 9H), 6.85 (d, J=9 Hz, 1H), 7.08 (d, J=9 Hz, 2 Hz, 1H), 7.22 (d, J=9 Hz, 2H), 7.42 (d, J=9 Hz, 2H), 7.48-7.61 (m, 28H), 7.96 (d, J=2 Hz, 1H); ¹³C NMR (150 MHz, CDCl₃) δ1.2 (CH₃), 127.08 (CH), 127.12 (CH), 127.2 (CH), 127.3 (CH), 127.4 (CH), 127.5 (CH), 127.7 (CH), 127.8 (CH), 129.6 (CH), 129.6 (CH), 129.9 (CH), 131.2 (CH), 132.8 (CH), 137.0 (4°), 137.5 (4°), 137.6 (4°), 137.75 (4°), 137.84 (4°), 137.9 (4°), 138.0 (4°), 138.1 (4°), 138.3 (4°), 138.75 (4°), 138.82 (4°), 142.3 (4°), 146.6 (4°); HRMS(ESI) m/z calcd for C₅₅H₄₄Si [M.]⁺: 756.3207. found 756.3182; not degraded or melted at 300° C. or more.

### Example 8

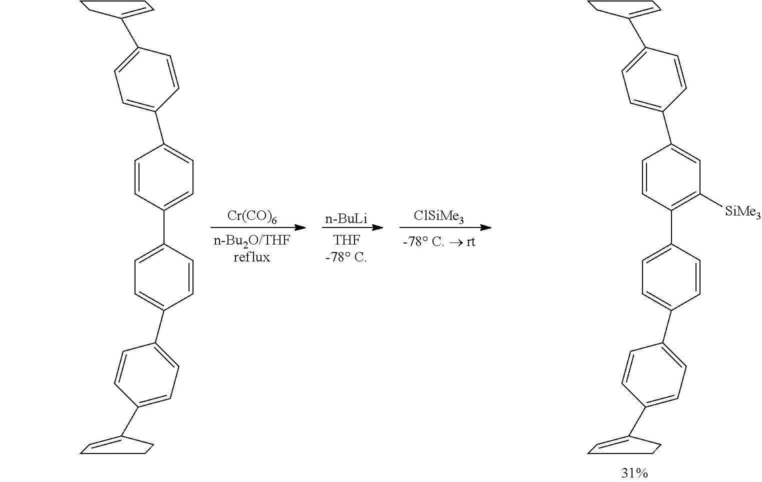

A magnetic stirring bar was placed in a J. Young® Schlenk flask, and [12]CPP (20.0 mg, 21.9 μmol), Cr(CO)₆ (9.9 mg, 43.8 μmol), dibutyl ether (15.3 mL), and THF (1.7 mL) were added to the flask. The reaction mixture was stirred at 160° C. for 1.5 hours in the dark and concentrated under reduced pressure. After the obtained product was dissolved in THF (5.0 mL), a hexane solution of 0.4 M n-butyllithium (110 μL, 44 μmol) was slowly added at −78° C., and the reaction mixture was stirred for 30 minutes in the dark (at this point, lithiated [12]CPP was obtained). Thereafter, chlorotrimethylsilane (100 μL, 780 μmol) was added to the reaction mixture, and the resulting reaction mixture was warmed to room temperature and stirred for 1 hour in the dark. The reaction mixture was quenched with water and exposed to air and room light to perform decomplexation for 24 hours. The obtained reaction mixture was concentrated under reduced pressure, and the crude product was purified by silica gel column chromatography (hexane/CHCl₃). As a result, the desired trimethylsilyl-[12]CPP was obtained as a yellow solid (6.7 mg, 31%), and the starting material [12]CPP was recovered (11.9 mg, 60%).

¹H NMR (600 MHz, CDCl₃) δ 0.32 (s, 9H), 6.96 (d, J=8 Hz, 1H), 7.23 (dd, J=8 Hz, 2 Hz, 1H), 7.30 (d, J=8 Hz, 2H), 7.52 (d, J=8 Hz, 2H), 7.58-7.66 (m, 40H), 7.98 (d, J=2 Hz, 1H); ¹³C NMR (150 MHz, CDCl₃) δ1.2 (CH₃), 127.0 (CH), 127.10 (CH), 127.13 (CH), 127.17 (CH), 127.20 (CH), 127.27 (CH), 127.29 (CH), 127.32 (CH), 127.36 (CH), 127.41 (CH), 127.44 (CH), 127.5 (CH), 127.6 (CH), 127.7 (CH), 129.1 (CH), 129.6 (CH), 131.8 (CH), 132.3 (CH), 137.6 (4°), 138.1 (4°), 138.2 (4°), 138.3 (4°), 138.36 (4°), 138.46 (4°), 138.50 (4°), 138.57 (4°), 138.64 (4°), 138.68 (4°), 138.70 (4°), 138.2 (4°), 139.4 (4°), 142.9 (4°), 147.3 (4°); HRMS (ESI) m/z calcd for C₅₅H₄₄Si [M.]⁺: 984.4146. found 984.4133; a melting point of 300° C. or more.

### Example 9

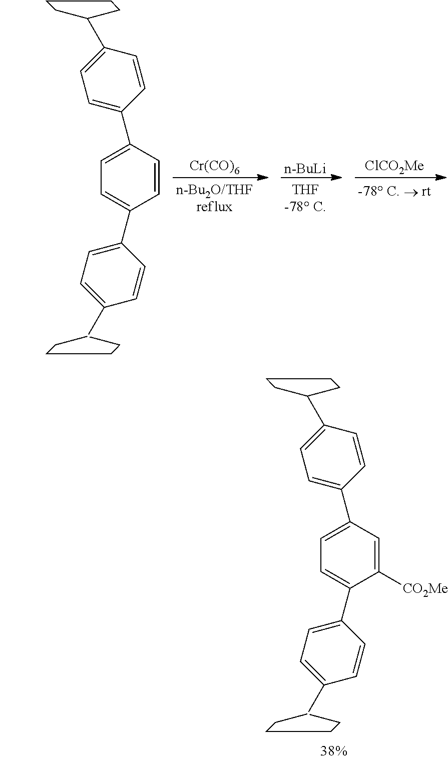

A magnetic stirring bar was placed in a J. Young® Schlenk flask, and [9]CPP (20.0 mg, 29.2 μmol), Cr(CO)₆ (7.2 mg, 31.7 μmol), dibutyl ether (9.0 mL), and THF (1.0 mL) were added to the flask. The reaction mixture was stirred at 160° C. for 1 hour in the dark and concentrated under reduced pressure. After the obtained product was dissolved in THF (5.0 mL), a hexane solution of 0.4 M n-butyllithium (150 μL, 60 μmol) was slowly added at −78° C., and the reaction mixture was stirred for 30 minutes in the dark (at this point, lithiated [9]CPP was obtained). Thereafter, methyl chloroformate (60 μL, 774 μmol) was added to the reaction mixture, and the resulting reaction mixture was warmed to room temperature and stirred for 1 hour in the dark. The reaction mixture was quenched with water and exposed to air and room light to perform decomplexation for 24 hours. The obtained reaction mixture was concentrated under reduced pressure, and the crude product was purified by silica gel column chromatography (hexane/CHCl₃). As a result, the desired carboxymethyl-[9]CPP was obtained as a yellow solid (8.2 mg, 38%), and the starting material [9]CPP was recovered (11.8 mg, 59%).

¹H NMR (600 MHz, CD₂Cl₂) δ3.85 (s, 3H), 7.04 (d, J=8 Hz, 1H), 7.25 (d, J=8 Hz, 2H), 7.33 (dd, J=8 Hz, 2 Hz, 1H), 7.47 (d, J=8 Hz, 2H), 7.51-7.62 (m, 28H), 8.38 (d, 2 Hz, 1H); ¹³C NMR (150 MHz, CD₂Cl₂) δ 52.5 (CH₃), 127.5 (CH), 127.60 (CH), 127.69 (CH), 127.77 (CH), 127.82 (CH), 127.86 (CH), 127.9 (CH), 128.1 (CH), 129.2 (CH), 129.3 (4°), 132.7 (CH), 134.3 (CH), 137.8 (4°), 137.9 (4°), 138.0 (4°), 138.26 (4°), 138.35 (4°), 138.47 (4°), 138.57 (4°), 138.64 (4°), 138.88 (4°), 140.5 (4°), 141.0 (4°), 168.6 (4°); HRMS (MALDI-TOF) m/z calcd for C₅₅H₃₇O [MH]⁺: 743.2945. found 743.2922. a melting point of 300° C. or more.

### Example 10

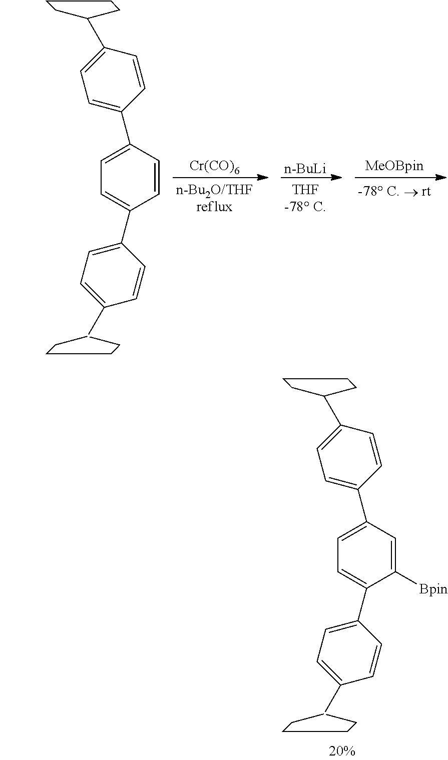

A magnetic stirring bar was placed in a J. Young® Schlenk flask, and [9]CPP (20.0 mg, 29.2 μmol), Cr(CO)₆ (7.2 mg, 31.7 μmol), dibutyl ether (9.0 mL), and THF (1.0 mL) were added to the flask. The reaction mixture was stirred at 160° C. for 1 hour in the dark and concentrated under reduced pressure. After the obtained product was dissolved in THF (5.0 mL), a hexane solution of 0.4 M n-butyllithium (150 μL, 60 μmol) was slowly added at −78° C., and the reaction mixture was stirred for 30 minutes in the dark (at this point, lithiated [9]CPP was obtained). Thereafter, methoxyboronic acid pinacol ester (50 μL, 305 μmol) was added to the reaction mixture, and the resulting reaction mixture was warmed to room temperature and stirred for 1 hour in the dark. The reaction mixture was quenched with water and exposed to air and room light to perform decomplexation for 24 hours. The obtained reaction mixture was concentrated under reduced pressure, and the crude product was purified by silica gel column chromatography (hexane/CHCl₃). As a result, the desired tetramethyldioxaboryl-[9]CPP was obtained as a yellow solid (4.7 mg, 20%), and the starting material [9]CPP was recovered (13.9 mg, 70%).

¹H NMR (600 MHz, CDCl₃) δ 1.34 (s, 12H), 7.03 (d, J=8 Hz, 1H), 7.24 (dd, J=8 Hz, 2 Hz, 1H), 7.30 (d, J=9 Hz, 2H), 7.43 (d, J=9 Hz, 2H), 7.50-7.55 (m, 28H), 8.20 (d, J=2 Hz, 1H); ¹³C NMR (150 MHz, CDCl₃) δ 24.6 (CH₃), 84.0 (4°), 127.08 (CH), 127.12 (CH), 127.18 (CH), 127.25 (CH), 127.36 (CH), 127.42 (CH), 127.44 (CH), 127.6 (CH), 127.7 (CH), 129.9 (CH), 131.3 (CH), 132.2 (CH), 132.5 (CH), 136.9 (CH), 137.66 (4°), 137.69 (4°), 137.73 (4°), 137.76 (4°), 137.82 (4°), 137.90 (4°), 137.92 (4°), 137.94 (4°), 137.99 (4°), 138.01 (4°), 138.4 (4°), 138.5 (4°), 140.9 (4°), 145.6 (4°); HRMS (ESI) m/z calcd for C₃H₄₇BO₂[M.]⁺: 810.3664. found 810.3653.

1. A cyclopolyarylene metal complex in which a metal tricarbonyl is coordinated to one benzene ring of a cyclopolyarylene compound.

2. The cyclopolyarylene metal complex according to claim 1, wherein the cyclopolyarylene compound is a cyclic compound in which at least one member selected from the group consisting of bivalent aromatic hydrocarbon groups and derivative groups thereof are continuously bonded.

3. The cyclopolyarylene metal complex according to claim 1, wherein the metal constituting the metal tricarbonyl is chromium, molybdenum, tungsten, iron, ruthenium, osmium, manganese, or rhenium.

4. A method for producing the cyclopolyarylene metal complex according to claim 1, the method comprising the step of (I) reacting a cyclopolyarylene compound with a metal compound represented by Formula (2):
M(CO)₃Yₘ,
wherein M is a metal atom; Y is the same or different, and each represents a ligand; m is an integer of 1 to 3.

5. The method according to claim 4, wherein the step (I) is performed in the presence of an ether solvent or a hydrocarbon solvent.

6. A metal-substituted cyclopolyarylene compound in which a metal atom is bonded to one carbon atom of one benzene ring of a cyclopolyarylene compound.

7. The metal-substituted cyclopolyarylene compound according to claim 6, wherein the metal atom is an alkali metal atom.

8. A method for producing a metal-substituted cyclopolyarylene compound, the method comprising the step of (II) reacting the cyclopolyarylene metal complex according to claim 1 with a metal compound.

9. The method according to claim 8, wherein the metal compound is an alkali metal compound.

10. The method according to claim 8, wherein the metal compound is an alkyllithium.

11. A functional-group-containing cyclopolyarylene compound in which a boronic acid group or an ester thereof, a silyl group, a carboxy group or an ester thereof, or a formyl group is bonded to one carbon atom of one benzene ring of a cyclopolyarylene compound.

12. A method for producing a functional-group-containing cyclopolyarylene compound, the method comprising the step of (III) reacting the metal-substituted cyclopolyarylene compound according to claim 6 with an electrophile.

13. The cyclopolyarylene metal complex according to claim 2, wherein the metal constituting the metal tricarbonyl is chromium, molybdenum, tungsten, iron, ruthenium, osmium, manganese, or rhenium.
